(19) United States
(12) Patent Application Publication
(10) Pub. No.: US 2023/0052494 A1
(43) Pub. Date: Feb. 16, 2023

(54) TRACKING TAGS FOR VENOUS CATHETERIZATION COMPLICATIONS

(71) Applicant: B. Braun Melsungen AG, Melsungen (DE)

(72) Inventors: Yasotha Padmanathan, Selangor (MY); Ka Hwee Kong, Penang (MY); Yie Miin Lee, Penang (MY); YongJi Fu, Penang (MY); Hui Kuun Teoh, Penang (MY); Noraini Jalil, Melaka (MY); Sharienna Sharman, Selangor (MY); Aik Aun Tan, Penang (MY)

(73) Assignee: B. Braun Melsungen AG, Melsungen (DE)

(21) Appl. No.: 17/793,699

(22) PCT Filed: Jan. 18, 2021
(86) PCT No.: PCT/EP2021/050968

§ 371 Date: Jul. 19, 2022

Related U.S. Application Data
(60) Provisional application No. 62/963,333, filed on Jan. 20, 2020.

(51) Int. Cl. A61M 5/168

(52) U.S. Cl. CPC A61M 5/16836; A61M 25/0017; A61M 2205/50; A61M 2205/3331; A61M 2205/3561; A61M 2205/3584; A61M 2205/3592; A61M 2230/50; A61M 2230/65

(57) ABSTRACT

A sensing system for sensing a potential complication at a venous catheter site. The system includes a sensor module for attachment at the site of the catheter. The sensor module includes a pressure sensor configured to generate pressure data representing measured pressure at the site of the catheter; a temperature sensor configured to generate temperature data representing measured temperature at the site of the catheter; and two pairs of bio impedance electrodes that generate bioelectrical signals representing bioelectrical activity at the site of venous catheter and a transmitter for transmitting the pressure, temperature data and bio impedance data. The system also includes a computing device configured to receive the response signal that includes the generated pressure, temperature and bio impedance data; and transmit the pressure temperature and bio impedance data to a user device for comparing the generated pressure temperature bio impedance data to threshold values indicative of intravenous complications.

### BACKGROUND OF THE INVENTION

Insertion of peripheral venous catheters (PVCs) is a commonly performed invasive procedure in a healthcare setting. More than 330 million PVCs are inserted every year in the United States alone. However, more than 40% of inserted PVCs develop complications requiring re-insertion of another PVC before a treatment can be completed. Among the most common complications associated with PVCs, phlebitis constituted about 7% of the incidence, occlusion about 15%, infiltrations about 19%, and catheter-related bloodstream infection (CRBSI) about 0.5%.

Current methods for detecting complications due to PVCs, such as an infection, include visual inspection and notification of pain by the patient. To confirm the presence of an infection at the PVC site, blood from the infected site and the infected catheter are typically cultured. The preparation of samples for the laboratory can be a laborious and time consuming process. Also, twenty-four to forty-eight hours may be needed before the results of the sampling can be made available to the clinician. If the complication (e.g., infection) is permitted to continue, it could become severe and require additional treatments and a longer stay in the hospital.

In view of the foregoing, it would be desirable to provide a device to detect complications at the PVC site.

### SUMMARY OF THE INVENTION

According to one aspect of the invention, a sensing system for sensing a potential complication at a site of a venous catheter inserted within a vein of a patient comprises:
(i) a sensor module configured for attachment at the site of the venous catheter, the sensor module including: a pressure sensor configured to generate pressure data representing measured pressure at the site of the venous catheter; a temperature sensor configured to generate temperature data representing measured temperature at the site of the venous catheter; two pairs of bio impedance sensors attached at sides of the pressure sensor for measuring a bio impedance signal from tissue at the site of the venous catheter; a transmitter coupled to the pressure sensor and the temperature sensor for receiving the pressure data and temperature data, the transmitter configured to receive an interrogation signal and produce a response signal in response to the interrogation signal, the response signal including the generated pressure, temperature and bio impedance data; and a biocompatible substrate configured to support the pressure sensor, the temperature sensor, the bio impedance sensors and the transmitter; and
(ii) a computing device including a non-transitory computer readable medium having stored thereon a program, wherein execution of the program of the non-transitory computer readable medium configures the computing device to: emit, with an antenna system of the computing device, the interrogation signal; receive, with the antenna system of the computing device, the response signal that includes the generated pressure, temperature and bio impedance data; and transmit the pressure, temperature and bio impedance data to a user device for comparing the generated pressure, temperature and bio impedance data to threshold values indicative of intravenous complications.

According to another aspect of the invention, a method for sensing a potential complication at a site of a venous catheter inserted within a vein of a patient comprising the steps of: positioning an intravenous complication sensing module over the site of the venous catheter, the intravenous complication sensing module configured to sense pressure, temperature and bio impedance at the site of the venous catheter; emitting, with an antenna system of a computing device, an interrogation signal; receiving, with the antenna system of the computing device, a response signal that includes the generated pressure, temperature data and bio impedance data; transmitting the generated pressure, temperature and bio impedance data to a user device; comparing, at the user device, the generated pressure, temperature and bio impedance data to threshold values indicative of intravenous complications; and alerting the user of the user device of the potential intravenous complication, with an output interface of the user device, when the compared generated pressure, temperature and bio impedance data exceeds one or more of the threshold values.

According to yet another aspect of the invention, a sensing module configured for attachment at a site of a venous catheter inserted within a vein of a patient comprises: a pressure sensor configured to generate pressure data representing measured pressure at the site of the venous catheter; a temperature sensor configured to generate temperature data representing measured temperature at the site of the venous catheter; a bio impedance sensor comprising bio impedance electrodes configured to generate bioelectrical data representing measured bio impedance at the site of the venous catheter; a transmitter coupled to the pressure sensor, the temperature sensor and bio impedance sensor, the transmitter configured to receive an interrogation signal and produce a response signal in response to the interrogation signal, the response signal including the generated pressure temperature data and bio impedance data; and a biocompatible substrate configured to support the pressure sensor, the temperature sensor, the bio impedance electrodes and the transmitter.

According to still another aspect of the invention, a sensing system for sensing a potential complication at a site of a venous catheter inserted within a vein of a patient comprises:
(a) a first temperature sensor configured to generate temperature data representing measured temperature at the site of the venous catheter;
(b) a second temperature sensor positioned at a pre-determined distance from the first temperature sensor and configured to measure a reference temperature at a second site on the patient; and
(c) a computing device including a non-transitory computer readable medium having stored thereon a program, wherein execution of the program of the non-transitory computer readable medium configures the computing device to compare the measured temperature with the reference temperature to determine the potential complication at the site of the venous catheter.

### BRIEF DESCRIPTION OF THE DRAWINGS

FIG. 1 depicts a sensor for detecting intravenous complications in a venous catheter that is inserted within a vein of a patient, and a complimentary wearable computing device that communicates with the sensor.

FIG. 2 depicts a cross-sectional view through the sensor and the arm of the patient.

FIG. 3 depicts a view of the sensor applied onto the skin and over a catheter.

FIG. 4 depicts a block diagram of the sensor and the complimentary wearable computing device.

FIG. 5 depicts a block diagram of the overall system for detecting intravenous complications.

FIGS. 6-8 depict block diagrams of alternative embodiments, each figure depicting a sensor and complimentary computing device.

FIG. 9 depicts an electronic health record (EHR) system structure.

### DETAILED DESCRIPTION OF THE INVENTION

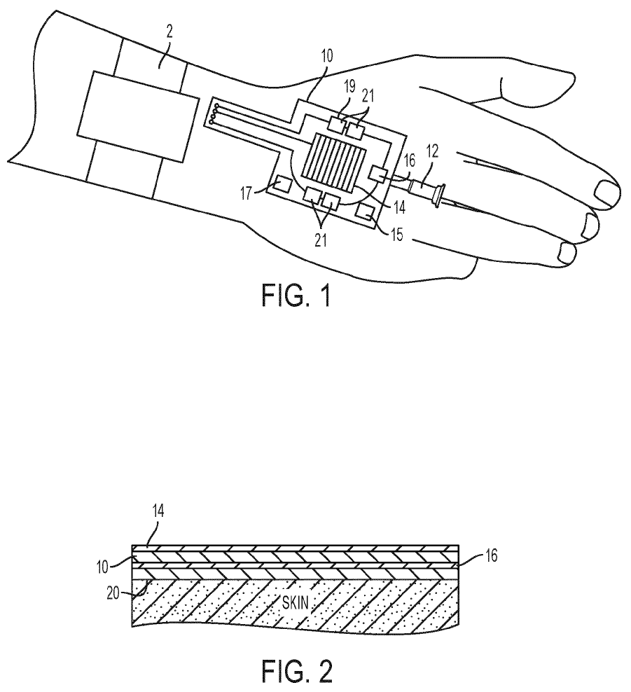

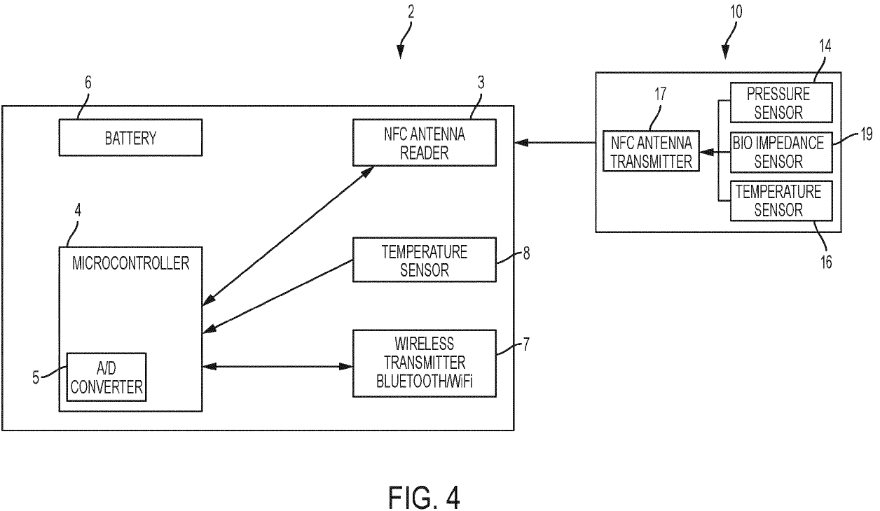

FIGS. 1 and 4 depicts a communication system comprising the sensor 10 and a complimentary wearable computing device 2. The sensor 10 (also referred to herein as a sensing module) is configured for detecting intravenous complications, and, more particularly, for sensing a potential complication at a site of a venous catheter 12 inserted within a vein of a patient. A gauze, bandage or dressing 9 is applied over the sensor 10 and the catheter 12.

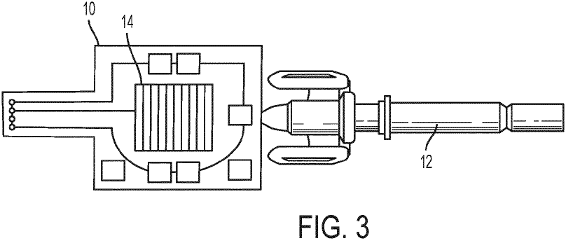

Referring now to FIGS. 1-3, the sensor 10 is an NFC tag in the form of a wearable patch that can be attached to the skin at the site of the venous catheter 12. The wearable patch may be a one-time wearable patch, or it may be reusable. The sensor 10 includes a pressure sensor 14, a temperature sensor 16, a bio impedance sensor 19 comprising two pairs of bio impedance electrodes 21, a microcontroller 15, an NFC antenna transmitter 17, and other components for organizing and transmitting data generated by the sensors. The components of the sensor 10 are mounted to a biocompatible substrate 20. The microcontroller 15 collects the data from the pressure sensor 14, the temperature sensor 16 and the bio impedance sensor 19 and transmits the data to the NFC antenna transmitter 17 for transmission. The microcontroller 15 includes a non-transitory computer readable medium having stored thereon a program. The sensor 10 is (optionally) a passive device that does not include a power source, however, the sensor 10 could alternatively be an active device having a power source.

The pressure sensor 14 is configured to generate pressure data representing measured pressure at the site of the venous catheter 12. The pressure sensor 14 measures swelling of the skin at the site of the venous catheter 12. A bend and stretching of the skin caused by swelling can be measured as a change in capacitance of the pressure sensor 14. The pressure sensor can be a screen printed flexible sensor on the biocompatible substrate 20.

A suitable screen printed flexible pressure sensor is described in U.S. Pat. No. 6,964,205, which is incorporated by reference herein in its entirety. The '205 patent describes a sensor for measuring a parameter applied to a surface. The sensor includes at least one substrate layer, a plurality of individual sensor elements operatively arranged with respect to the substrate layer, and a conductive trace disposed on the substrate layer. The conductive trace is electrically coupled to an individual sensor element and wraps around at least a portion of the sensor element in a spiral-like manner.

A suitable screen printed flexible pressure sensor is also described in Screen printed flexible pressure sensors skin. Khanet et al., 2014, Advanced Semiconductor Manufacturing Conference Proceedings, 219-224, which is incorporated by reference herein in its entirety. According to Khanet et al., the sensor comprises a polyvinylidene fluoride-trifluoroethylene P(VDF-TrFE) sandwiched between patterned metal layers. Bottom electrodes and P(VDF- TrFE) are printed on a 25 pm thick polyamide (PI) substrate. The top electrodes with force concentrator posts on backside are printed on a separate polyethylene terephthalate (PET) substrate. The two substrates are then adhered together. Each sensor module consists of a four by four sensor array, with each sensor having a 1 × 1 mm2 sensitive area.

Other examples of suitable pressure sensors are disclosed in U.S. Pat. Nos. 8138882, 9612690, and U.S. Pat. App. Pub. No. 2009/025681, each of which is incorporated by reference herein in its entirety.

The temperature sensor 16 is configured to generate temperature data representing temperature of the skin at the site of the venous catheter 12. The temperature sensor 16 may be a commercially available negative thermal coefficient (NTC) type thermistor, for example. A thermistor is a type of resistor whose resistance change is dependent on temperature. The thermistor can be a screen printed flexible sensor on the biocompatible substrate 20.

An example of a suitable screen printed temperature sensor is disclosed in U.S. Pat. App. Pub. No. 2017/0127944, which is incorporated by reference herein in its entirety. As described in the ’944 Publication, the NTC thermistor may include a substrate, a Si- C film printed on the substrate, and electrodes printed on the substrate for electrically connecting the Si- C film with control circuitry. The NTC thermistor may also include a first laminate arranged on the Si C film and the electrodes, and a second laminate arranged on a top surface of the first laminate for preventing moisture from penetrating into the NTC thermistor.

The bio impedance sensor 19 comprises two pairs of bio impedance electrodes 21. One pair of bio impedance electrodes 21 are stimulating electrodes that are configured to apply current to the skin or tissue, whereas the other pair of electrodes 21 are receiving electrodes that are configured to sense the impedance, resistance, or voltage applied to the skin. In use, current applied though the stimulating electrodes is transmitted to the skin/tissue. Swelling of the skin will cause a conductivity change, and the electrical impedance will be sensed by the receiving electrodes of the sensor 19. Variations in impedance affect the voltage detected by the receiving electrodes.

The biocompatible substrate 20 is configured to support the components of the sensor 10. The biocompatible substrate 20 is a thin and flexible biocompatible material, such as polyethylene, or other suitable biocompatible substrate such as those offered by 3M Medical Specialties, for example. An adhesive may be applied to an outer facing side of the substrate 20 for attachment to the patient’s skin.

The temperature sensor 16 is positioned near the catheter insertion site whereas the pressure sensor 14 covers a large surface area of the skin near the insertion site. The bio impedance electrodes 21 are placed at the sides of pressure sensor.

The NFC transmitter antenna 17 may be screen printed on the substrate 18 (or another substrate of the sensor 10). The NFC antenna transmitter 17 is connected to both the temperature sensor 16 and the pressure sensor 14, and receives measurement signals therefrom. The NFC antenna transmitter 17 includes a coil that receives/derives electrical power from the wearable computing device 2, as will be described hereinafter.

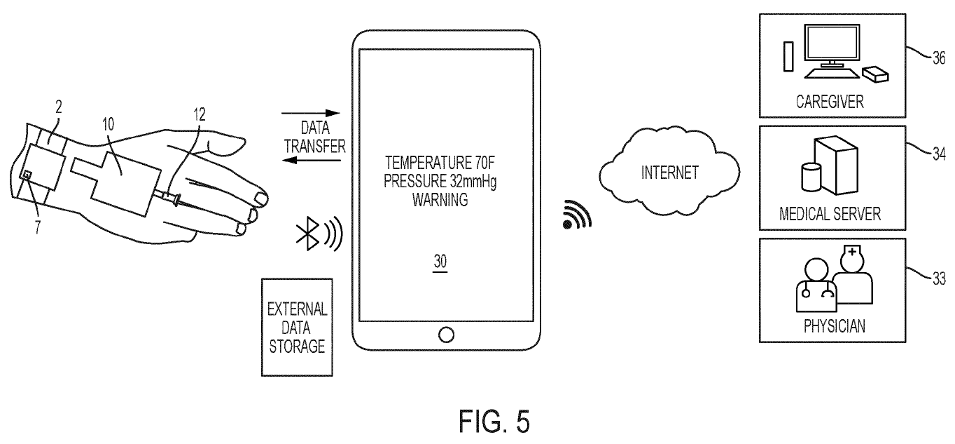

Referring back to FIGS. 1 and 5, the computing device 2 is mounted on a band that is wearable around the user’s arm or leg (for example) at a location adjacent the sensor 10. The band could be replaced by a wearable patch, for example. The computing device 2 includes a NFC antenna reader 3, a microcontroller 4, an analog to digital converter (ADC) 5, a battery (or, more generally, a power supply) 6, a wireless transmitter 7, a temperature sensor 8, a driver, memory, and an alarm system. The microcontroller 4 includes a non- transitory computer readable medium having stored thereon a program. The battery 6 supplies power to the NFC antenna reader 3, the microcontroller 4, the ADC 5, the alarm system and the wireless transmitter 7.

The temperature sensor 8 captures the normal body temperature of the user. The temperature data is primarily used to compare with the local temperature data received from sensor 10 at the insertion site of the catheter. The temperature sensor 8 can be used as a threshold value to monitor the temperature fluctuation at the insertion site of the catheter.

Provided that the computing device 2 and the sensor 10 are in close proximity, a coil of the NFC antenna reader 3 delivers electrical power to the coil of the NFC antenna transmitter 17 of the sensor 10 via inductive coupling so as to power the NFC antenna transmitter 17. The NFC antenna transmitter 17 may deliver power to the pressure sensor 14 and the temperature sensor 16 if those components require electrical power for operation. Alternatively, the sensor 10 and the computing device 2 may be physically and releasably connected together such that data is transmitted between the sensor 10 and the computing device 2 by a wired connection.

In operation, the program of the non-transitory computer readable medium of the microcontroller 4 is executed such that the NFC antenna reader 3 of the computing device 2 emits an interrogation signal to the NFC antenna transmitter 17 of the sensor 10. The interrogation signal is received by the NFC antenna transmitter 17 of the sensor 10. In response, the NFC antenna transmitter 17 of the sensor 10, which receives analog measurement data from the temperature sensor 16, the pressure sensor 14 and the bio impedance sensor 19, transmits that analog measurement data to the computing device 2 by way of NFC communication (or, alternatively, a direct wired connection). The NFC antenna reader 3 of the computing device 2 receives the analog measurement data from the NFC antenna transmitter 17 of the sensor 10. The NFC antenna reader 3 transmits the analog measurement data to the microcontroller 4. The microcontroller 4 then compares the temperature reading with the reading of the temperature sensor 8 to determine the normal body temperature range at a distance away from the insertion site of the catheter. A multiplexer of the microcontroller 4 arranges the analog measurement data into a single line, as is known in the art. The ADC 5 of the microcontroller 4 converts the multiplexed analog pressure, temperature and bio impedance data to digital data, as is known in the art. The microcontroller 4 sends the digital data to the wireless transmitter 7. As is shown with respect to FIG. 5, the wireless transmitter 7 on the computing device 2 transmits the digital data via Bluetooth or Wi-Fi to a smartphone 30 and/or external data storage.

FIG. 5 depicts a block diagram of the overall system for detecting intravenous complications. The overall system includes the sensor 10 which may be applied to the patient at the site of a catheter 12, the computing device 2 which is worn by the patient at a location adjacent the sensor 10, and a user device in the form of the smartphone 30 (or other electronic device, such as a tablet or computer). The transmitter 7 of the computing device 2 communicates wirelessly with the smartphone 30 via Bluetooth or Wi-Fi. The smartphone 30 communicates wirelessly over the Internet with a physician’s portal or device 33, a server 34 and/or a caregiver’s computer 36.

The smartphone 30 (or an application device loaded on the smartphone) is configured to track the pressure, temperature and bio impedance data and determine whether the skin pressure, bio impedance and/or temperature at the site of the catheter 12 has increased above pre-defined threshold values. More particularly, the smartphone 30 compares the generated pressure, temperature and bio impedance data to threshold values indicative of intravenous complications. If the skin pressure, bio impedance and/or temperature at the site of the catheter 12 has increased above a pre-defined threshold value, then the smartphone 30 alerts the user (clinician) via the GUI of the smartphone 30 that a possible infection exists at the site of the catheter 12. The computing device 2 may also transmit an audible or visual alert via the alarm system of the computing device 2. Thereafter, the clinician can respond accordingly.

As an optional step, the smartphone 30 wirelessly transmits the pressure, temperature and bio impedance data to a physician’s portal 33, the server 34 and/or the caregiver’s computer 36 via the internet. The pressure, temperature and bio impedance data may be stored in the physician’s portal 33, the server 34 or the caregiver’s computer 36. Also, physician’s portal 33, the server 34 or the computer 36 may be configured to determine whether the skin pressure and/or temperature at the site of the catheter 12 has increased/decreased compared with a pre-defined threshold value. Besides identifying the relevant complications at the injection site, the smartphone 30, the physician’s portal 33, the server 34 and/or the caregiver’s computer 36 can include a database for storing the pressure, temperature and bio impedance data.

According to one method of using the system of FIG. 5 for sensing a potential complication at a site of a venous catheter inserted within a vein of a patient, the method includes the step of positioning an intravenous complication sensing module (i.e., sensor 10) over the site of the venous catheter 12. The sensor 10 may be positioned along the vein where a capillary of the catheter was inserted. The positioned sensor 10 may be adhered to skin of the patient. The sensor 10 is configured to sense pressure, temperature and the bio impedance values at the site of the venous catheter, as described above. The computing device 2 is then positioned in close proximity to the sensor 10, e.g., less than 20 cm.

The method further comprises emitting an interrogation signal using an antenna system of the computing device 2, or, alternatively, connected via a wire. The sensor 10 is configured to receive the interrogation signal from the computing device 2. Power is delivered to the components of the sensor 10 via inductive coupling between the coils of the NFC antenna transmitter 17 and the NFC antenna reader 3, or, alternatively, via a cable connection. The sensors 14 and 16 then transmit analog input signals to the NFC antenna transmitter 17, as described above, and the NFC antenna transmitter 17 then transmits the signal to the NFC antenna reader 3 on the computing device 2. The NFC antenna reader 3 then transmits the signal to the microcontroller 4, which includes the ADC 5 that digitizes the analog signal. Microcontroller 4 then compares the received value with a pre-defined threshold value for both temperature and pressure readings. The microcontroller 4 transmits the digitized signal to the wireless transmitter 7. The wireless transmitter 7 then transmits a signal containing the digitally converted pressure, bio impedance and temperature data.

The method further comprises receiving, with the antenna system of the user device 30, the digitally converted pressure, temperature and bio impedance data. The method further comprises comparing the pressure, temperature and bio impedance data to threshold values indicative of intravenous complications, and alerting the user of the user device 30 of the potential intravenous complication, with an output interface of the user device 30, when the compared data exceeds one or more of the threshold values indicating a potential problem at the insertion site.

This is a continuous process where the data can be read at any point in time when there is a trigger of values above threshold levels. For example, during a condition where an onset inflammation is developing at the puncture site. The intravenous complication sensing module 10 could indicate the relevant parameters to the clinicians to take an immediate step before the condition becomes severe.

Various inventive aspects described herein provide advantages such as, inter alia:
1. A continuous monitoring solution to detect early complication on site, which may lead to reduced hospital stays.
2. Preventing removal of an existing catheter and re-insertion of a new catheter due to false alarm caused by visual inspection only.
3. Proper documentation process as the IV site is monitored carefully with continuous reading of data.

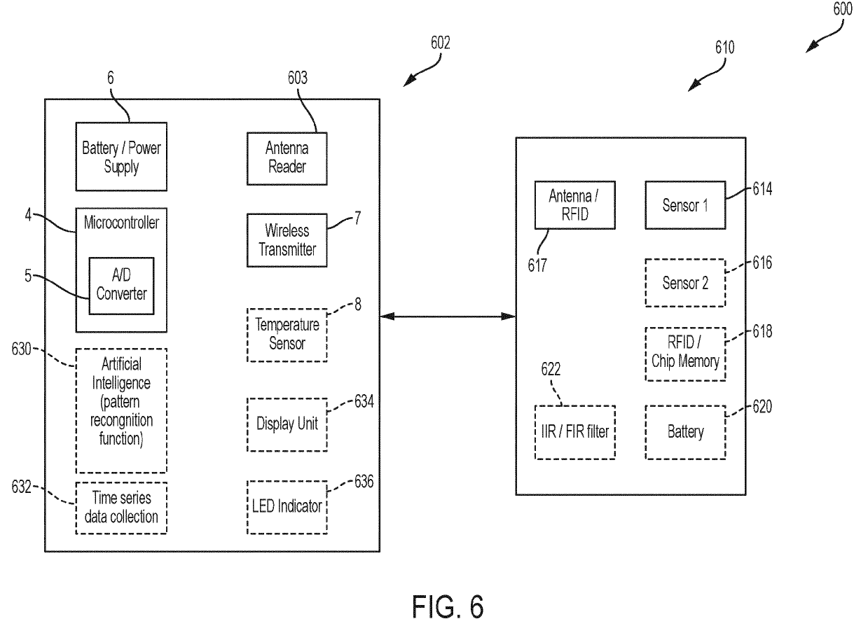

FIGS. 6-8 depict block diagrams of alternative embodiments, each figure depicting a sensor and complimentary computing device. These embodiments are related to the embodiment shown in FIG. 4, and only the differences therebetween will be described hereinafter.

FIG. 6 depicts a system 600 comprising a sensor assembly 610 (sensor 610 hereinafter) and complimentary computing device 602. The optional components of the system 600 are bounded by broken lines. The system 600 utilizes radiofrequency identification (RFID) for communications between the sensor 610 and the computing device 602. For clarification purposes, RFID is the process by which items are uniquely identified using radio waves, and NFC is a specialized subset within the family of RFID technology. NFC is a branch of High-Frequency (HF) RFID. A UHF frequency may be utilized.

The sensor 610 may be a passive RFID tag in the form factor of a wearable patch that can be attached to the skin at the site of the venous catheter 12 for detecting intravenous complications, and, more particularly, for sensing a potential complication at the site of a venous catheter 12 inserted within a vein of a patient. The sensor 610 may also be a wearable and removable device having a band, for example, that is attached to an appendage of a user. The sensor 610 may be used for other purposes. For example, the sensor 610 may be connected to a medical device, such as an infusion pump, for monitoring one or more conditions, such as fluid delivery, of the infusion pump.

If the sensor 610 is connected to an infusion pump, for example, the sensor 610 may report important information to assist inventory management (e.g., intravenous product, manufacturer, expiration date or other product information). A healthcare worker can link a patient ID number to the sensor 610, if so desired. The sensor assembly 610 includes a first sensor 614, a second sensor 616, an RFID antenna 617, an RFID chip having memory 618, a battery 620, and a filter 622 having either finite Impulse Response (FIR) or Infinite Impulse Response (HR). Each sensor 614 and 616 may be a temperature sensor, a pressure sensor, a bio-impedance sensor, an optical or fiber optic sensor, a capacitance sensor or a moisture sensor, for example. The temperature sensor may be a current output temperature sensor, a voltage output temperature sensor, a resistance temperature detector, a diode temperature sensor or a digital output temperature sensor, for example. Each sensor 614 and 616 could alternatively be a fluid rate sensor (for infusion), a body temperature sensor, a fluid temperature sensor (for infusion), a counter sensor for timing of catheter indwelling time frame, or a motion sensor.

If the sensor 614 is not a temperature sensor, then the sensor 616 may be omitted. If sensor 614 is a temperature sensor, then the sensor 616 may also be a temperature sensor. If both sensors 614 and 616 are temperature sensors, then the first sensor 614 may be applied to a patient directly at the insertion site, for example, and the second sensor 616 may be applied at a proximal reference site located at a pre-determined distance from the first sensor 614. The second sensor 616 is configured to provide a reference temperature for comparative purposes with the readings of the first sensor 614. The first sensor 614 may be positioned, for example, on the either on or near the at a horn of the catheter at the insertion site. The second sensor 616 may be positioned at a proximal location, such as on the patient at a pre-determined distance from the sensor 614, e.g., 3 cm, 4 cm, or any other desired distance. The second sensor 616 may be aligned with the first sensor 614 along the infusion fluid flow path. The second sensor 616 may be positioned laterally of the first sensor 614 or on opposite sides of the appendage of the patient (e.g., if the first sensor 614 is positioned on top of the hand, then the second sensor 616 could be positioned on either the side or at the palm of the same hand). The sensors 614 and 616 could be located on different appendages (e.g., one sensor on the arm and the other sensor on the leg, or sensors at the same location on different arms). The sensors 614 and 616 may be positioned above the indwelling catheter. The sensors 614 and 616 may be positioned or either the same or different dressings. The sensor 616 may be positioned on a separate band that is applied around the user’s wrist, for example.

The computer device 602 comprises an antenna reader 603, the microcontroller 4 having the analog to digital converter 5, the battery 6, the wireless transmitter 7, a temperature sensor 8, an artificial intelligence (AI) unit 630 having a pattern recognition function, a time series data collection unit 632, a display unit 634, and an LED indicator 636.

The antenna reader 603 may be an RFID antenna reader, an NFC antenna reader or both an NFC antenna reader and an RFID antenna reader. NFC may be used for shorter distances, while RFID may be used for greater distances. If the antenna 617 is an NFC antenna, then the reader 603 will be an NFC antenna reader. And, if the antenna 617 is an RFID antenna, then the reader 603 will be an RFID antenna reader.

The controller 4 is configured to interpret data received from the sensor 610, analyze that data and make decisions on the existence of intravenous complications or a low flow rate at the infusion rate, for example, using a threshold value, threshold ratio, trends, mean rate, change rate or frequency comparison, for example. The AI unit 630 (or the controller 4) is configured to recognize patterns, filter out irrelevant data, and reduce the possibility of false alarms (e.g., pressure drops due to patient movement, or temperature drops due to patient hand washing or entering cold room).

In use, a coil of the antenna reader 603 delivers an interrogation signal (in the form of electrical power) to the coil of the RFID/antenna 617 of the sensor 610 via inductive coupling (for example) so as to power the RFID antenna 617. The RFID/antenna 617 may deliver power to the sensors 614 and 616 if those components require electrical power for operation. The sensors 614 and 616 take measurements, and transmit the measurements back to the device 602 via the RFID/antenna 617 and the antenna reader 603, as was described above with respect to the other embodiments. This process can occur at pre determined intervals.

The device 602 may be positioned in a hospital, for example, such as a patient room, a corridor, on a patient bed, either on or embedded within an infusion pump, for example. The device 602 could function as a patient portal in the form of a mobile application, and the mobile application could transmit signals to the sensor 610 to activate the sensor 610, and, thereafter, capture patient related information transmitted by the sensor 610.

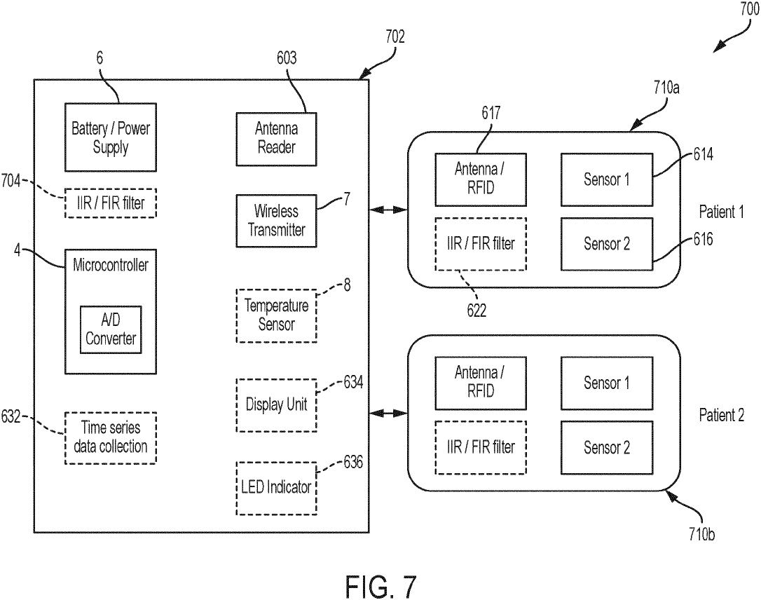

FIG. 7 depicts a system 700 comprising two sensor assemblies 710a and 710b (referred to either collectively or individually as sensor(s) 710) and a complimentary computing device 702. One sensor 710a is associated with a first patient, and the other sensor 710b is associated with a second patient. Although only two sensors 710 are shown it should be understood that any number of sensors 710 may be employed in the system 700. The optional components of the system 700 are bounded by broken lines. The system 700 is substantially similar to the system 600 and only the differences therebetween will be described.

Each sensor 710 includes the first sensor 614, the second sensor 616, the RFID antenna 617, and the filter 622. The first and second sensors 614 and 616 are preferably temperature sensors, however, this may vary. The computer device 702 comprises the NFC/RFID antenna reader 603, the microcontroller 4 having the analog to digital converter 5, the battery 6, the wireless transmitter 7, the temperature sensor 8, an IIR/FIR filter 704, the time series data collection unit 632, a display unit 634, and an LED indicator 636. The device 702 is configured to process information transmitted by both sensors 710.

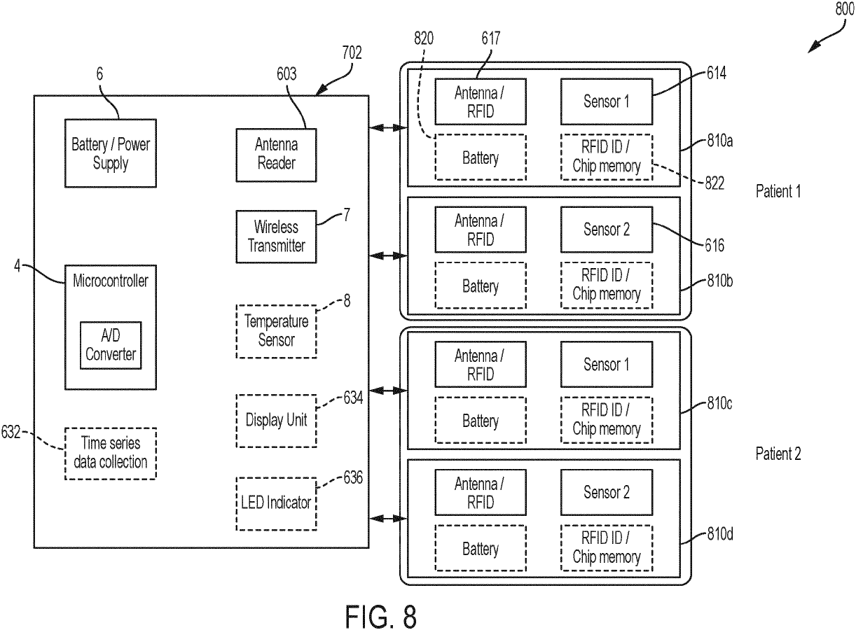

FIG. 8 depicts a system 800 comprising four sensors 810a-810d (referred to either collectively or individually as sensor(s) 810) and the complimentary computing device 702 of FIG. 7. Two sensors 810a and 810b are either applied to or associated with a first patient, whereas the other two sensors 810c and 810d are either applied to or associated with a second patient. Although only four sensors 810 are shown it should be understood that any number of sensors 810 may be employed in the system 800. The optional components of the system 800 are bounded by broken lines. The system 800 is substantially similar to the system 700 and only the differences therebetween will be described.

Each sensor 810 includes one of the first sensor 614 and the second sensor 616, the RFID antenna 617, a battery 820, and an RFID ID chip 822 having memory. The first and second sensors 614 and 616 are preferably temperature sensors, however, this can vary. For example, patient 1 wears two sensors 810a and 810b on either the same or different appendages, as was described above. The sensor 810a may be applied at the site of a venous catheter, whereas the sensor 810b may be mounted at a proximal location for reference purposes, and the sensor 616 of the sensor 810b provides an ambient or reference temperature.

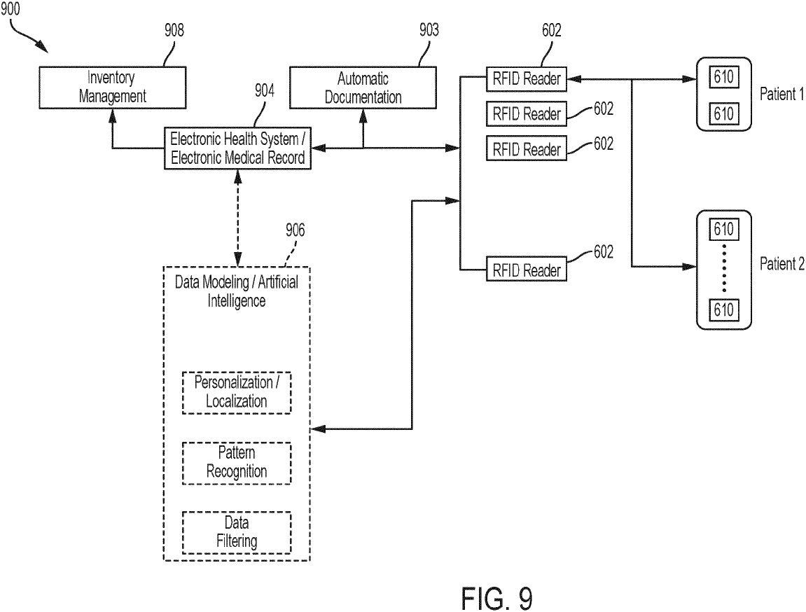

FIG. 9 depicts an electronic health record (EHR) system structure 900. The system 900 comprises a series of sensors (such as sensor 610, but may be a different sensor) worn by multiple patients. Each sensor 610 communicates with one or more RFID readers (such as device 602, but may be a different device). The devices 602 transmit raw data to a documentation system 903. The devices 602 also transmits data to an electronic health system 904. The electronic health system 904 (optionally) communicates with a data modeling and AI system 906. The AI system 906 performs pattern recognition, data filtering and other AI functions based, at least in part, on personal and location based for each patient. The electronic health system 904 then transmits data to an inventory management system 908.

Although the invention is illustrated and described herein with reference to specific embodiments, the invention is not intended to be limited to the details shown. Rather, various modifications may be made in the details within the scope and range of equivalents of the claims and without departing from the invention.

1. A sensing system for sensing a potential complication at a site of a venous catheter inserted within a vein of a patient, the sensing system comprising:
(i) a sensor module configured for attachment at the site of the venous catheter, the sensor module including:
(a) a pressure sensor configured to generate pressure data representing measured pressure at the site of the venous catheter;
(b) a temperature sensor configured to generate temperature data representing measured temperature at the site of the venous catheter;
(c) a bio impedance sensor comprising two pairs of bio impedance electrodes that are together configured to generate bio impedance data comprising bio-electrical signals representing bioelectrical activity at the site of the venous catheter;
(d) a transmitter coupled to the sensors and the bio impedance electrodes, the transmitter configured to receive an interrogation signal and produce a response signal in response to the interrogation signal, the response signal including the generated pressure, temperature and bio impedance data; and
(e) a biocompatible substrate configured to support the pressure sensor, the temperature sensor, the bio impedance sensor and the transmitter; and
(ii) a computing device including a non-transitory computer readable medium having stored thereon a program, wherein execution of the program of the non-transitory computer readable medium configures the computing device to:
(a) emit, with an antenna system of the computing device, the interrogation signal;
(b) receive, with the antenna system of the computing device, the response signal that includes the generated pressure, temperature and bio impedance data; and
(c) transmit the generated pressure, temperature and bio impedance data to a user device for comparing the generated pressure, temperature and bio impedance data to threshold values indicative of intravenous complications.

2. The system of claim 1, wherein the computing device comprises:
a microcontroller,
a temperature sensor,
a near field communication (NFC) reader coupled to the microcontroller for receiving the pressure, temperature and bio impedance data and modulating the pressure, temperature and bio impedance data onto a response signal, and
wherein the antenna system of the computing device is a wireless transmitter coupled to the microcontroller for transmitting the response signal to the user device.

3. The system according to claim 1, wherein the pressure sensor, the temperature sensor, and the bio impedance sensor are positioned on the same layer of the biocompatible substrate.

4. The system according to claim 1, wherein the antenna system is a wireless transmitter that is configured to emit signals via Bluetooth or Wi-Fi.

5. A method for sensing a potential complication at a site of a venous catheter inserted within a vein of a patient, the method comprising:
positioning an intravenous complication sensing module over the site of the venous catheter, the intravenous complication sensing module configured to sense pressure, temperature and bio impedance values at the site of the venous catheter;
emitting, with an antenna system of a computing device, an interrogation signal;
receiving, with the antenna system of the computing device, a response signal that includes the sensed pressure, temperature and bio impedance values;
transmitting the sensed pressure, temperature and bio impedance values to a user device;
comparing, at the user device, the sensed pressure, temperature and bio impedance values to threshold pressure, temperature and bio impedance values indicative of intravenous complications; and
alerting the user of the user device of the potential intravenous complication, with an output interface of the user device, when one or more of the sensed pressure, temperature and bio impedance values exceeds one or more of the threshold values.

6. The method of claim 5, wherein the positioning step comprises:
positioning the intravenous complication sensing module along the vein where a capillary of the catheter was inserted; and
adhering the positioned intravenous complication sensing module to skin of the patient.

7. A sensing module configured for attachment at a site of a venous catheter inserted within a vein of a patient, the sensing module comprising:
a pressure sensor configured to generate pressure data representing measured pressure at the site of the venous catheter;
a temperature sensor configured to generate temperature data representing measured temperature at the site of the venous catheter;
a bio impedance sensor comprising two pairs of bio impedance electrodes that are together configured to generate bio impedance data comprising bio-electrical signals representing bioelectrical activity at the site of the venous catheter;
a transmitter coupled to the pressure sensor, the bio impedance sensor and the temperature sensor, the transmitter configured to receive an interrogation signal and produce a response signal in response to the interrogation signal, the response signal including the generated pressure, bio impedance and temperature data; and
a biocompatible substrate configured to support the pressure sensor, the temperature sensor, the bio impedance sensor and the transmitter.

8. The sensing module of claim 7, wherein the biocompatible substrate includes an adhesive configured to adhere the sensing module to the site of the venous catheter.

9. The sensing module according to claim 7, wherein the pressure sensor, the temperature sensor, and the bio impedance sensor are positioned on a same layer of the biocompatible substrate.

10. A sensing system for sensing a potential complication at a site of a venous catheter inserted within a vein of a patient, the sensing system comprising:
(a) a first temperature sensor configured to generate temperature data representing measured temperature at the site of the venous catheter;
(b) a second temperature sensor positioned at a pre-determined distance from the first temperature sensor and configured to measure a reference temperature at a second site on the patient; and
(c) a computing device including a non-transitory computer readable medium having stored thereon a program, wherein execution of the program of the non-transitory computer readable medium configures the computing device to compare the measured temperature with the reference temperature to determine the potential complication at the site of the venous catheter.

11. The sensing system of claim 10, wherein the first and second temperature sensor are either coupled to one transmitter or are coupled to separate transmitters, wherein the or each transmitter is configured to receive an interrogation signal and produce a response signal in response to the interrogation signal, the response signal including generated temperature data from one or both of the temperature sensors, and wherein a sensing module comprises one transmitter and at least one of the temperature sensors.

12. The sensing system according to claim 10 , wherein the sensing module comprises a first transmitter of the separate transmitters and the first temperature sensor, and a second sensing module comprises a second transmitter of the separate transmitters and the second temperature sensor.

13. The sensing system according to claim 10 , wherein the sensing module comprises said one transmitter and the first and second temperature sensors.

14. The sensing system according to claim 10 , wherein the computing device is detached from the sensing module, and the computing device is configured to:
(a) emit, with an antenna system of the computing device, the interrogation signal; and
(b) receive, with the antenna system of the computing device, the response signal that includes the generated temperature data from at least one of the temperature sensors.

15. The sensing system according to claim 10 , wherein the sensing module is associated with a first patient, and the sensing system comprises a second sensing module associated with a second patient, wherein the computing device is configured to receive signals from the first and second sensing modules.

16. The sensing system according to claim 10 , wherein the computing device is configured to transmit, with an antenna system of the computing device, the response signal that includes the generated temperature data from each temperature sensor to a documentation system or an electronic health system.

17. The sensing system according to claim 10 , wherein the predetermined distance is at least 3 cm.
